(19) United States
(12) Patent Application Publication
(10) Pub. No.: US 2001/0025142 A1
(43) Pub. Date: Sep. 27, 2001

(54) Medical examination apparatus with means for acquiring patient and/or apparatus movements

(76) Inventors: Gerd Wessels, Effeltrich (DE); Karl Barth, Hoechstadt (DE)

Correspondence Address: SCHIFF HARDIN & WAITE, 6600 SEARS TOWER, 233 S WACKER DR, CHICAGO, IL, 60606-6473, US

(21) Appl. No.: 09/867,179

(22) Filed: May 29, 2001

Related U.S. Application Data
(62) Division of application No. 09/089,547, filed on Jun. 3, 1998, now abandoned.

(30) Foreign Application Priority Data
Jun. 13, 1997 (DE) 19725137.4

(51) Int. Cl. A61B 6/00

(52) U.S. Cl. 600/425; 600/585

(57) ABSTRACT

Movements of the patient or of a medical examination apparatus, for example of a C-arm, are acquired by sensors that supply corresponding signals. The sensors can be sensitive to magnetic fields and acquire the magnetic fields of magnet elements or the movement acquisition can ensue optically or acoustically.

### BACKGROUND OF THE INVENTION

1. Field of the Invention

The present invention is directed to a medical examination apparatus for conducting an examination or an interventional surgical procedure, of the type having moving apparatus components.

2. Description of the Prior Art

Movements of the patient and apparatus components movements during the image acquisition can lead to motion artifacts in a medical examination apparatus for generating images of an examination subject, for example an x-ray apparatus, a computed tomography apparatus or a nuclear magnetic resonance tomography apparatus. In particular, such artifacts in the acquisition of image data can occur at different points in time, for example in digital subtraction angiography, in the image synthesis from exposures at several points in time or given different aspect angles of the measurement unit. Intracorporeal dislocations of organs, for example of the brain when the skull is opened, can also lead to spatial displacement of a body process which is to be subjected to therapy.

U.S. Pat. No. 5,566,220 discloses a computed tomography apparatus wherein a sensor is provided for the acquisition of focus movements, the output signals of this sensor serving the purpose of compensating different sensitivities in the detector channels. Patient movements and movements of mechanical apparatus parts can lead to image artifacts in this computed tomography apparatus, since no compensation measures for this purpose are undertaken. German OS 42 10 121 discloses an x-ray diagnostic arrangement wherein the x-ray tube is pulsed, and the pulse frequency is set dependent on the heart activity of the patient. This known arrangement can only compensate motion unsharpness due to heart activity. Other motion unsharpness due to patient movements or movements of apparatus parts are not acquired and can also not be compensated by means of the pulse repetition rate of the x-radiation.

### SUMMARY OF THE INVENTION

The present invention is to provide a medical examination apparatus for generating images of an examination subject wherein image artifacts due to patient and/or mechanical apparatus movements are largely avoided.

The above object is achieved in accordance with the principles of the present invention in a medical examination apparatus having an image data acquisition system and image electronics for generating images of an examination subject from the image data, one or more sensors for identifying patient movements and/or movements of apparatus components during the acquisition of the image data, the sensors supplying signals to the image electronics identifying these movements, and wherein the image electronics undertakes a correction of the image data, dependent on the detected movements, so that motion artifacts in the image are avoided are minimized.

In the inventive apparatus, a sensor system, for example an electromagnetic or optical sensor system is provided, that acquires patient and apparatus movements during the pick-up of image data. Location-variant image data can be corrected in real time given movements due to the output signals of this sensor system. A re-correction by electronic shift of the image information for achieving congruency of two successively registered images with motion artifacts (pixel script) is not necessary.

### DESCRIPTION OF THE DRAWINGS

FIG. 1 shows a medical examination apparatus for explaining the invention.

FIG. 2 shows the support table of the apparatus according to FIG. 1 with the patient for explaining the acquisition of movements.

FIG. 3 shows the processing of the information derived from the acquired movements.

FIG. 4 shows a configuration of a medical examination apparatus in accordance with the invention for surgical interventions at the brain.

### DESCRIPTION OF THE PREFERRED EMBODIMENTS

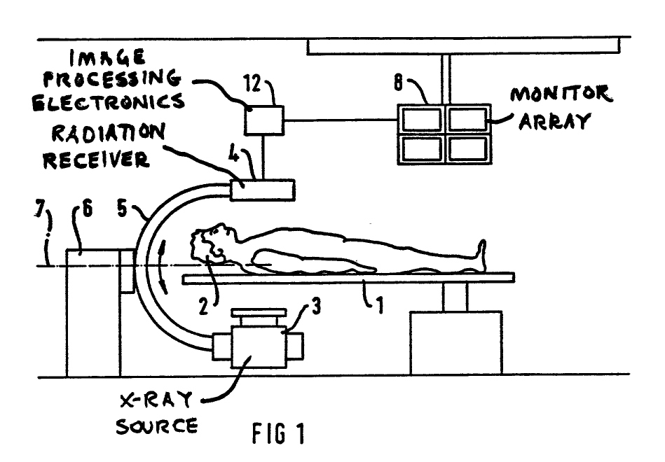

FIG. 1 shows a support table 1 on which a patient 2 lies. An x-ray source 3 and a radiation receiver 4 are provided for producing medical images, namely x-ray images given the exemplary embodiment of. The x-ray source 3 and the radiation receiver 4 are secured to a C-arm 5 that is seated at a base 6 so as to be adjustable in the direction of its circumference, and is seated so as to be rotatable around a system axis 7. The radiation receiver 4 can, for example, be a surface detector composed of a matrix of detector elements whose output signals are supplied to an image processing electronics 12 for generating x-ray images given various angular positions of the C-arm 5. The image reproduction ensues at a monitor array 8.

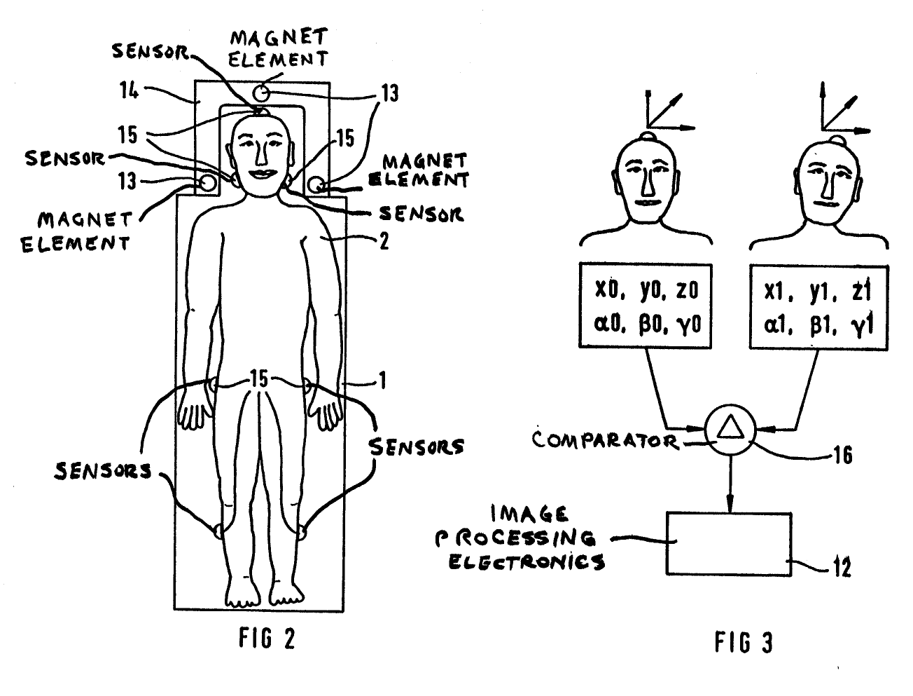

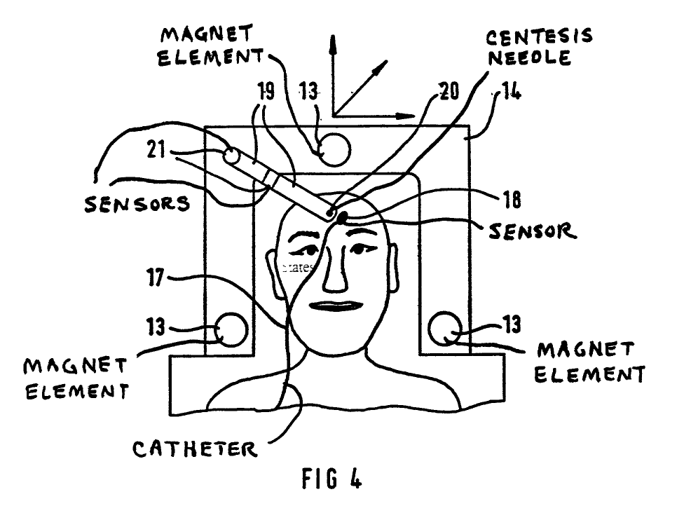

FIG. 2 shows that magnetic elements 3 (not shown in FIG. 1) acting as transmitters are provided in the region of the bearing table 1, these magnet elements 13 generating magnetic fields that are acquired by sensors 15 at the patient. The output signals of the sensors 15 correspond to the spatial coordinates x0, y0, z0 or to the angular coordinates α0, β0, γ0, as well as the values x1, y1, z1; α1, β1, γ1 (spatial coordinates after unwanted movement on the part of the patient). These output signals are supplied to a comparator 16 according to FIG. 3 that acquires dislocations of the patient 2 or of the support table 1, or of the C-arm 5, i.e. acquires patient and/or apparatus movements, and supplies corresponding difference signals to the image processing electronics 12 for image correction.

FIG. 4 shows an exemplary embodiment wherein a catheter 17 is introduced for a brain operation and is placed in the therapy zone, this catheter 17 carrying a sensor 18 at its tip for detecting the magnetic fields of the magnet elements 13. When the skull is opened, the brain mass and, thus, the spatial position of the catheter 17 can shift. This dislocation is acquired by the sensor 18 that supplies corresponding correction signals to the image electronics 12. The deviation compared to the original position can be acquired given possible modification of the spatial position of the catheter tip, for example due to movements.

The invention is described in conjunction with the acquisition of patient and apparatus movements with the assistance of magnetic fields and magnetic field sensors. Within the scope of the invention, this acquisition can also ensue wireless in some other way, for example optically or acoustically.

FIG. 4 also shows an articulated arm 19 on the frame 14 that is seated so as to be adjustable relative to the frame 14 so that a puncture (centesis) needle 20 arranged at its free end can be three-dimensionally adjusted in space. As a result, the puncture needle 20 can be directed exactly to the desired location, for example to the location of the tip of the catheter 17. At its articulations, the articulated arm 19 contains sensors 21 that supply data corresponding to the spatial position of the puncture needle 20. Information about the spatial position of the catheter tip can be acquired via the magnet elements 13 and the sensor 18. The puncture needle 20 can be directed exactly to the position of the sensor 18, i.e. to the desired location, by comparing these two sets of information.

Although modifications and changes may be suggested by those skilled in the art, it is the intention of the inventors to embody within the patent warranted hereon all changes and modifications as reasonably and properly come within the scope of their contribution to the art.

We claim as our invention:

1. A medical examination apparatus comprising:
means for obtaining image data from an examination subject;
image processing means, supplied with said image data, for producing an image of said examination subject from said image data, said image being subject to motion artifacts due to movement of said examination subject during acquisition of said image data;
sensor means for identifying movements of said patient and for generating a sensor output signal corresponding to said movements; and
said image processing means including means, supplied with said sensor output signal, for correcting said image of said examination subject dependent on said sensor output signal for minimizing motion artifacts in said image due to movements of said subject.

2. A medical examination apparatus as claimed in claim 1 further comprising a patient table adapted to receive said examination subject thereon during acquisition of said image data, and wherein said sensor means includes at least one magnetic element disposed at said table, said magnetic element generating a magnetic field, and at least one magnetic field sensor adapted for attachment to said examination subject, said magnetic field sensor generating said sensor output signals.

3. A medical examination apparatus comprising:
data acquisition means for acquiring image data from an examination subject;
at least one movable apparatus component whose movement influences said image data;
image processing means, supplied with said image data, for generating an image of said examination subject from said image data, said image being subject to motion artifacts due to movement of said apparatus component;
sensor means for generating a sensor output signal dependent on movement of said apparatus component; and
said image processing means including means, supplied with said sensor output signal, for correcting said image data dependent on said sensor output signal for minimizing said motion artifacts in said image.

4. A medical examination apparatus as claimed in claim 3 wherein said sensor means comprises at least one magnetic element which generates a magnetic field, and wherein said apparatus component comprises a catheter having a catheter tip, and said sensor means further including a magnetic field sensor disposed at said catheter tip.

5. A medical examination apparatus as claimed in claim 3 wherein said movable apparatus component comprises an interventional instrument and means for mounting said interventional instrument relative to an examination subject so that said interventional instrument is freely adjustable in space, position sensor means for generating position data identifying a spatial position of said interventional instrument, and said apparatus further comprising means for aligning said interventional instrument by comparing said data from said position sensor means to said sensor output signal of said sensor means.
